(19) United States
(12) Patent Application Publication
(10) Pub. No.: US 2021/0059886 A1
(43) Pub. Date: Mar. 4, 2021

(54) APPARATUS FOR TENSIONING SPINE AND METHOD OF TENSIONING SPINE

(71) Applicant: CBH LTD., Jeonju-si, Jeollabuk-do (KR)

(72) Inventor: Jong Kyu YOON, Gwangyang-si, Jeollanam-do (KR)

(21) Appl. No.: 16/769,928

(22) PCT Filed: Nov. 29, 2018
(86) PCT No.: PCT/KR2018/015000

§ 371 Date: Nov. 10, 2020

(30) Foreign Application Priority Data
Dec. 5, 2017 (KR) 10-2017-0166228

(51) Int. Cl. A61H 1/02

(52) U.S. Cl. CPC A61H 1/0222; A61H 2201/1619; A61H 2201/1604

(57) ABSTRACT

Provided is a spinal traction device and a spinal traction method for spinal traction, and the spinal traction device includes: an upper body support unit configured to support an upper body of a person and having at least one spine pressing unit installed on one surface thereof; a lower body support unit configured to support a lower body of the person and having a moving means so as to move away from or close to the upper body support unit; and a controller configured to adjust a movement of the lower body support unit. The spinal traction device has a technical feature in which at least one spine pressing unit is in close contact with and supports the spine of the upper body when the lower body support unit moves.

### TECHNICAL FIELD

The present invention relates to a spinal traction device and a spinal traction method, and more particularly, to a spinal traction device and a spinal traction method, which support a spine with air bags when fixing a body and stretching the spine.

### BACKGROUND ART

Typically, a person, who walks or sits, often maintains his/her waist in a slightly curved state rather than in a completely upright state. Due to this motion, muscles, muscle, fasciae, and various types of ligaments, at a waist and a back, are excessively stretched and intervertebral discs (discs) are greatly compressed, especially, in a sedentary lifestyle. Modern people spend not less than 8 hours a day while sitting, and the pressure applied to their waists while they are sitting is as much as 8 times or more the pressure while they are lying. Therefore, the flexibility of a spinal joint significantly deteriorates due to the excessive load consistently applied to the waist, and back pain frequently occurs. This symptom causes great discomfort during activity, and also affects a deterioration in growth of minors.

Accordingly, various traction/correction devices for spinal therapy have been proposed. Regarding the spinal therapy devices in the related art, there have been developed methods of treating lesion sites in the spine, such as a method of applying acupressure to the spine and stimulating the spine, a method of stretching and treating the waist and the neck, a method of massaging the spine with heat, and a method of twisting the spine to the left and the right.

However, these spinal therapy devices have a disadvantage in that it is impossible to treat the spine in a stepwise manner in accordance with a curvature state of the spine because a treatment means is limited.

As a document related to the present invention, there is Korean Patent No. 10-1615183 (registered on Apr. 19, 2016).

### Document of Related Art

(Patent Document 1) KR 10-1615183 B1 (2016 Apr. 19.)

### DISCLOSURE

### Technical Problem

The present invention has been made in an effort to solve the above-mentioned problems, and an object of the present invention is to provide a spinal traction device and a spinal traction method, which are capable of stretching a spine in a state in which a curvature of the spine is maintained as the spine is supported by air bags when fixing a part of a body and stretching the spine.

### Technical Solution

In order to achieve the above-mentioned object, a spinal traction device according to the present invention includes: an upper body support unit configured to support an upper body of a person and having at least one spine pressing unit installed on one surface thereof; a lower body support unit configured to support a lower body of the person and having a moving means so as to move away from or close to the upper body support unit; and a controller configured to adjust a movement of the lower body support unit, in which at least one spine pressing unit is in close contact with and supports a spine of the upper body when the lower body support unit moves.

Particularly, the upper body support unit may include: a head support unit configured to support a head of the person; and a thoracic support unit configured to support thoracic vertebrae of the person, in which at least one of the head support unit and the thoracic support unit is provided with a moving means so that the head support unit and the thoracic support unit move away from or close to each other, and in which at least one of the head support unit and the thoracic support unit is provided with the spine pressing unit, such that the spine pressing unit is in close contact with and supports the spine (cervical vertebrae) of a neck part of the upper body when at least one of the head support unit and the thoracic support unit moves.

Particularly, the spine pressing unit may include a plurality of air bags, and the plurality of air bags may be disposed in a longitudinal direction of the spine.

Particularly, the plurality of air bags may be disposed in the longitudinal direction of the spine, and at least one air bag may be disposed at each of the left and right sides based on the spine.

Particularly, the controller may adjust to twist the spine to the left or the right by adjusting the amount of air to be supplied to the air bag at the left side and the air bag at the right side.

Particularly, the spinal traction device may further include a lateral upper body pressing unit installed to be movable in a direction of the spine of the upper body and configured to adjust a curvature of the spine in order to press a lateral portion of the upper body supported on the upper body support unit.

Particularly, the spinal traction device may include first and second lumbar traction tubes installed at an end of the upper body support unit and an end of the lower body support unit, respectively, and configured to be supplied with air from a compressor in order to press lumbar vertebrae.

Particularly, the upper body support unit may include a cervical/thoracic relief unit that supports the cervical vertebrae and the thoracic vertebrae of the spine.

Particularly, the cervical/thoracic relief unit may include an upper cervical relief unit configured to support the upper cervical vertebrae adjacent to the head of the person; a lower cervical relief unit positioned in a direction of the lumbar vertebrae with respect to the upper cervical relief unit and configured to support the lower cervical vertebrae; and a thoracic relief unit disposed between the lower cervical relief unit and the first lumbar traction tube.

Particularly, the upper body support unit may include: a head support unit configured to support the head of the person and on which the upper cervical relief unit is installed; and a thoracic support unit structured to be separated from the head support unit so as to be spaced apart from the head support unit, in which the lower cervical relief unit is installed at one side of the thoracic support unit, the first lumbar traction tube is installed at the other side of the thoracic support unit, and the thoracic relief unit is installed between the lower cervical relief unit and the first lumbar traction tube.

Particularly, the spinal traction device may further include a spine scanning unit installed on the upper body support unit, mounted to be movable in accordance with a curvature of the spine in a direction from the cervical vertebrae to the lumbar vertebrae of the spine, and configured to detect the state of the spine.

A spinal traction device according to the present invention includes: an upper body support unit configured to support an upper body of a person; a lower body support unit configured to support a lower body of the person and having a moving means so as to move away from or close to the upper body support unit; and a controller configured to adjust a movement of the lower body support unit, in which at least one of the upper body support unit and the lower body support unit is installed to be movable upward and downward, such that at least one of the upper body support unit and the lower body support unit moves upward or downward to press a spine of the upper body when the lower body support unit moves.

A spinal traction method according to the present invention includes: a spine scanning step of detecting a state of a spine by a spine scanning unit installed on an upper body support unit for supporting an upper body and configured to be movable in accordance with a curvature of the spine in a direction from cervical vertebrae to lumbar vertebrae; an air supply step of supplying air into at least one air bag installed on the upper body support unit by receiving information about the curvature of the spine transmitted from the spine scanning unit; and a lumbar traction step of stretching the lumbar vertebrae by moving a lower body support unit away from the upper body support unit in the state in which the air is supplied into the air bag.

### Advantageous Effects

The spinal traction device according to the present invention may stretch the spine in the state in which a curvature of the spine is maintained as the spine is supported by the air bags when fixing a part of the body and stretching the spine.

In addition, according to the spinal traction device according to the present invention, the spine may be supported by supplying different amounts of air into the plurality of air bags, and as a result, it is possible to treat the spine in a stepwise manner in accordance with a curvature state of the spine.

In addition, the spinal traction device according to the present invention has an advantage in that it is possible to improve efficiency in treating spinal stenosis and scoliosis because it is possible to twist the spine to the left or the right by individually supplying different amounts of air to the air bags disposed at the left and right sides based on the spine.

### DESCRIPTION OF DRAWINGS

FIG. 1 is a conceptual view illustrating a spinal traction device according to an exemplary embodiment of the present invention.

FIG. 2 is a schematic side view illustrating the spinal traction device illustrated in FIG. 1.

FIG. 3 is a block diagram of the spinal traction device illustrated in FIG. 1.

FIG. 4 is a partial perspective view illustrating a spine scanning unit according to another exemplary embodiment of the present invention.

FIG. 5 is a partial conceptual view illustrating a spinal traction device according to still another exemplary embodiment of the present invention.

FIG. 6 is a perspective view illustrating a spinal traction device according to yet another exemplary embodiment of the present invention.

FIG. 7 is a flowchart of a spinal traction method according to the exemplary embodiment of the present invention.

### BEST MODE

The present invention may be variously modified and may have various exemplary embodiments, and particular exemplary embodiments illustrated in the drawings will be described in detail below. However, the description of the exemplary embodiments is not intended to limit the present invention to the particular exemplary embodiments, but it should be understood that the present invention is to cover all modifications, equivalents and alternatives falling within the spirit and technical scope of the present invention. In the description of the drawings, similar reference numerals are used for similar constituent elements.

The terms such as “first,” “second,” “A,” “B,” and other numerical terms may be used herein only to describe various elements, but these elements should not be limited by these terms. These terms are used only to distinguish one constituent element from another constituent element. For example, a first constituent element may be named a second constituent element, and similarly, the second constituent element may also be named the first constituent element, without departing from the scope of the present invention. The term “and/or” includes any or all combinations of a plurality of the related and listed items.

When one constituent element is described as being “connected” or “coupled” to another constituent element, it should be understood that one constituent element can be connected or coupled directly to another constituent element, and an intervening constituent element can also be present between the constituent elements. When one constituent element is described as being “connected directly to” or “coupled directly to” another constituent element, it should be understood that no intervening constituent element is present between the constituent elements.

The terminology used herein is used for the purpose of describing particular exemplary embodiments only and is not intended to limit the present invention. Singular expressions include plural expressions unless clearly described as different meanings in the context. In the present application, it will be appreciated that terms “including” and “having” are intended to designate the existence of characteristics, numbers, steps, operations, constituent elements, and components described in the specification or a combination thereof, and do not exclude a possibility of the existence or addition of one or more other characteristics, numbers, steps, operations, constituent elements, and components, or a combination thereof in advance.

Unless otherwise defined, all terms used herein, including technical or scientific terms, have the same meaning as commonly understood by those skilled in the art to which the present invention pertains. The terms such as those defined in a commonly used dictionary should be interpreted as having meanings consistent with meanings in the context of related technologies and should not be interpreted as ideal or excessively formal meanings unless explicitly defined in the present application.

Hereinafter, preferred exemplary embodiments according to the present invention will be described in detail with reference to the accompanying drawings.

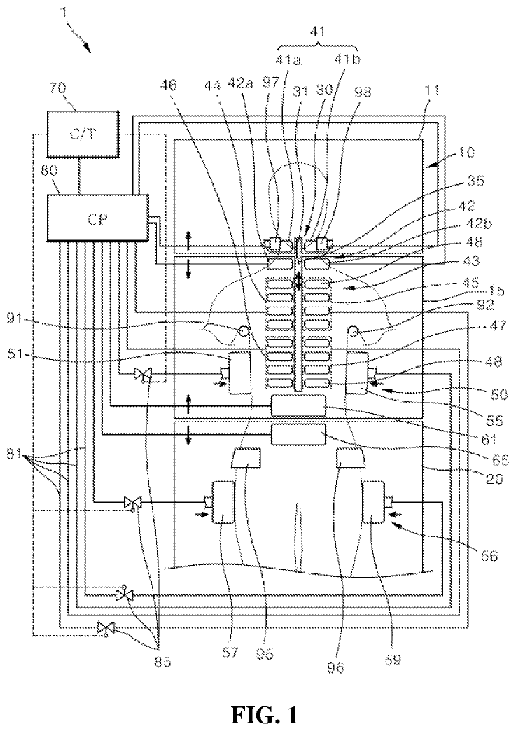

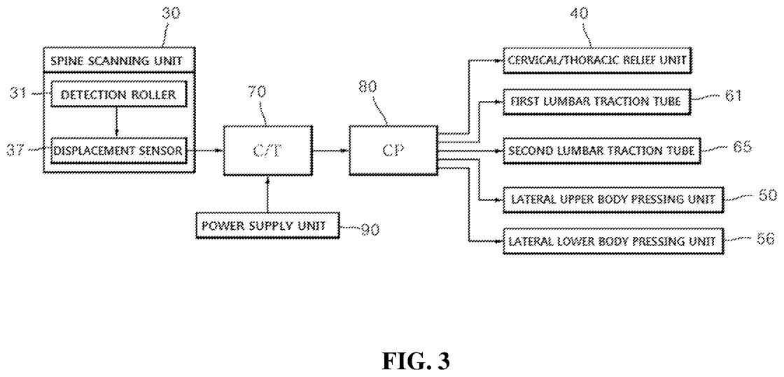

A human spine is broadly divided into cervical vertebrae, thoracic vertebrae, and lumbar vertebrae. Referring to FIGS. 1 to 3, a spinal traction device 1 according to the present invention includes: an upper body support unit 10 configured to support an upper body of a person and having at least one spine pressing unit installed on one surface thereof; a lower body support unit 20 configured to support a lower body of the person and having a moving means so as to move away from or close to the upper body support unit 10; and a controller 70 configured to adjust a movement of the lower body support unit 20. At least one spine pressing unit may be in close contact with and support the spine of the upper body when the lower body support unit 20 moves.

In addition, as necessary, the spinal traction device according to the present invention may further include a spine scanning unit 30, a lateral upper body pressing unit 50, a lateral lower body pressing unit 56, first and second lumbar traction tubes 61 and 65, the controller 70, a compressor 80, air supply pipes 81, on-off valves 85, and a power supply unit (not illustrated).

In the present invention, the spine pressing unit is a constituent element configured to press the spine including the cervical vertebrae, the thoracic vertebrae, and the lumbar vertebrae, and the spine pressing unit presses the spine as the upper body support unit 10 or the lower body support unit 20 moves. In the present invention, the spine is stretched as the upper body support unit 10 or the lower body support unit 20 moves. In this case, the spine pressing unit presses the spine, which may fix the spine to improve an effect of stretching the spine and may maintain a curvature of the spine to improve an effect of correcting the spine.

The spine pressing unit may be formed in the form of an air bag that may be supplied with air from the compressor 80. Otherwise, the spine pressing unit may be formed in a shape having no vacant space therein.

As specifically described in the following exemplary embodiment, the spine pressing unit according to the present invention may include at least one of an upper cervical relief unit 41, a lower cervical relief unit 42, air bags 48, the first lumbar traction tube 61, and the second lumbar traction tube 62.

The upper body support unit 10 supports the upper body of the person, and the lower body support unit 20 supports the lower body of the person. The upper body support unit 10 and the lower body support unit 20 are structured to be separated so that a spacing distance therebetween may be increased in an extension direction of the spine based on the lumbar vertebrae. The upper body support unit 10 and the lower body support unit 20 may be installed based on the lumbar vertebrae so that the lumbar vertebrae may be stretched in opposite directions.

In addition, the upper body support unit 10 may include: a head support unit 11 configured to support the person's head; and a thoracic support unit 15 configured to support the person's thoracic vertebrae. In this case, a moving means may be provided on at least one of the head support unit 11 and the thoracic support unit 15, such that the head support unit 11 and the thoracic support unit 15 move away from or close to each other. The spine pressing unit may be provided on at least one of the head support unit 11 and the thoracic support unit 15, such that the spine pressing unit may be in close contact with and support a spine of a neck part of the upper body, that is, the cervical vertebrae when at least one of the head support unit 11 and the thoracic support unit 15 moves.

Further, the upper body support unit 10 may include: the head support unit 11 configured to support the person's head and on which the upper cervical relief unit 41 of the cervical/thoracic relief unit is installed; and the thoracic support unit 15 structured to be separated from the head support unit 11 so as to be spaced apart from the head support unit 11. The lower cervical relief unit 42 of the cervical/thoracic relief unit 40 is installed at one side of the thoracic support unit 15, the first lumbar traction tube 61 is installed at the other side of the thoracic support unit 15, and a thoracic relief unit 43 is installed between the lower cervical relief unit 42 and the first lumbar traction tube 61.

In addition, as illustrated in FIG. 1, the upper body support unit 10 may include first and second upper body movement preventing bars 91 and 92 configured to support both armpits of the upper body in order to prevent the upper body from moving in a movement direction of the lower body support unit 20 when the upper body support unit 10 moves so that the spacing distance between the upper body support unit 10 and the lower body support unit 20 is increased. The upper body support unit 10 may include first and second neck support units 97 and 98 configured to press or support lateral portions of the neck.

In addition, the lower body support unit 20 may include first and second lower body fixing units 95 and 96 configured to fix the lower body in order to prevent the lower body from moving in a movement direction of the upper body support unit 10 when the lower body support unit 20 moves so that the spacing distance between the lower body support unit 20 and the upper body support unit 10 is increased.

Although not illustrated, the spinal traction device according to the present invention has the moving means that allows the upper body fixing unit 10 and the lower body fixing unit 20 to move away from or close to each other.

The moving means may include: rack gears (not illustrated) provided at lower sides of the upper body fixing unit 10 and the lower body fixing unit 20, respectively, and extending in a direction in which the upper body fixing unit and the lower body fixing unit move away from or close to each other; pinion gears (not illustrated) configured to engage with the rack gears, respectively, and rotate; and rotary motors (not illustrated) configured to rotate the pinion gears. However, the structure, which enables the upper body fixing unit and the lower body fixing unit to move away from or close to each other, is not limited to the rack gear and the pinion gear, and the spacing distance may be adjusted by using a hydraulic or pneumatic cylinder.

The moving means may of course be applied to a structure that increases or decreases the spacing distance between the head support unit 11 and the thoracic support unit 15 of the upper body support unit 10.

The spine scanning unit 30 is mounted on the upper body support unit 10 so as to be movable in accordance with the curvature of the spine in a direction from the cervical vertebrae to the lumbar vertebrae, detects a scoliosis state or a stenosis state of the spine, and transmits the detection result to the controller 70 to be described below.

The spine scanning unit 30 has a guide unit 35, a detection roller 31, a lifting support unit 32, and a displacement sensor 37.

The guide unit 35 is penetratively formed between the plurality of air bags 48 of the cervical/thoracic relief unit 40 to be described below which is symmetrically arranged in the extension direction of the spine, and the guide unit 35 communicates with an internal space (not illustrated) of the upper body support unit 10. The guide unit 35 extends in the direction from the upper body support unit 10 to the lower body support unit 20.

The detection roller 31 is movable in the extension direction of the guide unit 35. The detection roller 31 is installed to be retractable in a direction from the guide unit 35 to the spine in accordance with the curvature of the spine supported on the upper body support unit 10. The detection roller 31 rotates in a state of being in contact with the skin of the upper body corresponding to the spine.

The detection roller 31 is rotatably installed on the lifting support unit 32 which is retractable in the direction from the guide unit to the spine direction. The lifting support unit 32 may be mounted to be movable upward or downward by a hydraulic cylinder (not illustrated), and a rack gear (not illustrated) and a pinion gear (not illustrated), which engage with each other so that the detection roller 31 may move, together with the lifting support unit 32, in the extension direction of the guide unit 35, may be installed at an end of the lifting support unit. In addition, a drive motor (not illustrated) may of course be installed for the rotational motion of the rack gear.

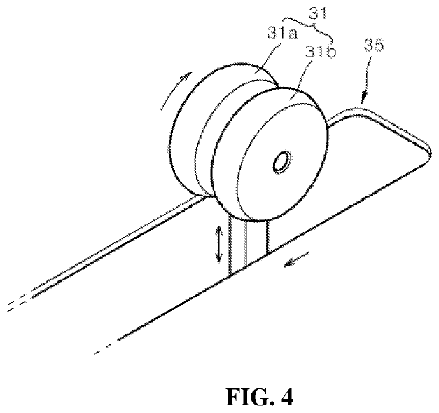

In addition, as illustrated in FIG. 4, the detection roller 31 may be divided into a first detection roller 31a and a second detection roller 31b which are in parallel with each other in the extension direction of the guide unit and spaced apart from each other at a predetermined interval.

This is advantageous in that the spine is seated between the first and second detection rollers 31a and 31b spaced apart from each other, such that the spine may be prevented from being withdrawn from the first and second detection rollers 31a and 31b when the first and second detection rollers 31a and 31b rotate.

Meanwhile, unlike that illustrated in FIG. 1, the guide unit 35 may extend in the extension direction of the spine so that the detection roller 31 is movable only along the thoracic support unit 15.

The displacement sensor 37 measures a position of the detection roller 31 and a distance by which the detection roller 31 moves while rotating, and the displacement sensor 37 transmits information to the controller 70 to be described below. The displacement sensor 37 may be any of a rotation sensor configured to detect a rotational speed and a rotation angle of the detection roller 31, an optical displacement sensor, and a motion recognition sensor, or a plurality of sensors may be applied in combination. Depending on types of applied sensors, the displacement sensor may be installed on an outer circumferential surface of the detection roller 31 and may be in contact with the skin corresponding to the spine. The displacement sensor may be installed inside the detection roller 31. The displacement sensor 37 may consistently detect information about positions that vary over time when the detection roller 31 rotates.

The spine scanning unit 30 may collect information based on the number of operations and an operation interval of a switch (not illustrated) which is connected to the displacement sensor 37 and operates when a change in position occurs in accordance with a curve of the spine when the detection roller 31 rotates along the spine.

The cervical/thoracic relief unit 40 has the plurality of air bags 41a, 41b, 42a, 42b, and 48 arranged on the upper body support unit 10 in the extension direction of the spine so as to be symmetrical based on the spine scanning unit 30 in order to support the cervical vertebrae and the lumbar vertebrae, and air is supplied into the plurality of air bags 41a, 41b, 42a, 42b, and 48. The amount of air to be supplied into the respective air bags 48 may be individually controlled by the controller 70 so that the spine may be twisted to the left and the right, thereby supporting the spine.

The cervical/thoracic relief unit 40 includes the upper cervical relief unit 41 configured to support the upper cervical vertebrae adjacent to the person's head, the lower cervical relief unit 42 positioned in a direction of the lumbar vertebrae with respect to the upper cervical relief unit and configured to support the lower cervical vertebrae, and the thoracic relief unit 43 positioned between the lower cervical relief unit 42 and the first lumbar traction tube 61 and configured to support the thoracic vertebrae of the spine.

The upper cervical relief unit 41 has the first and second upper cervical relief tubes 41a and 41b which are the air bags positioned symmetrically at the left and right sides based on the spine scanning unit and into which the air is injected. The lower cervical relief unit 42 has the first and second lower cervical relief tubes 42a and 42b which are the air bags positioned symmetrically at the left and right sides based on the spine scanning unit and into which the air is injected.

Referring to FIG. 1, the thoracic relief unit 43 may include: a first thoracic relief unit 44 and a third thoracic relief unit 46 positioned at the left side of the spine scanning unit 30 based on the spine scanning unit 30 and arranged to be sequentially spaced apart from one another in the direction from the cervical vertebrae to the lumbar vertebrae; and a second thoracic relief unit 45 and a fourth thoracic relief unit 47 positioned at the right side of the spine scanning unit 30 and installed at the positions corresponding to the first thoracic relief unit 44 and the third thoracic relief unit 46, respectively, in the direction from the cervical vertebrae to the lumbar vertebrae.

The first and second thoracic relief units 44 and 45 are installed to be spaced apart from the first and second lower cervical relief tubes 42a and 42b at predetermined distances in the direction of the lumbar vertebrae, and the plurality of air bags 48 may be installed symmetrically.

The third and fourth thoracic relief units 46 and 47 are installed to be spaced apart from the first and second thoracic relief units 43 and 44 at predetermined distances in the direction of the lumbar vertebrae, and the plurality of air bags 48 may be installed symmetrically.

In the cervical/thoracic relief unit 40, together with the upper cervical relief unit 41, the lower cervical relief unit 42, and the first and second lumbar traction tubes 61 and 65 to be described below, the amount of air to be supplied into the plurality of air bags 48 may be controlled by the controller so that the spine may have an S-shaped curvature or the lumbar vertebrae and the cervical vertebrae may have a C-shaped curvature.

In addition, the cervical/thoracic relief unit 40 may twist and support the spine under control of the controller 70 by supplying the air into the air bags of the first thoracic relief unit 44 and the fourth thoracic relief unit 47 and by restricting the supply of air into the air bags of the second thoracic relief unit 45 and the third thoracic relief unit 46. On the contrary, the cervical/thoracic relief unit may twist and support the spine under control of the controller 70 by restricting the supply of air into the air bags of the first thoracic relief unit 44 and the fourth thoracic relief unit 47 and by supplying the air into the air bags of the second thoracic relief unit 45 and the third thoracic relief unit 46.

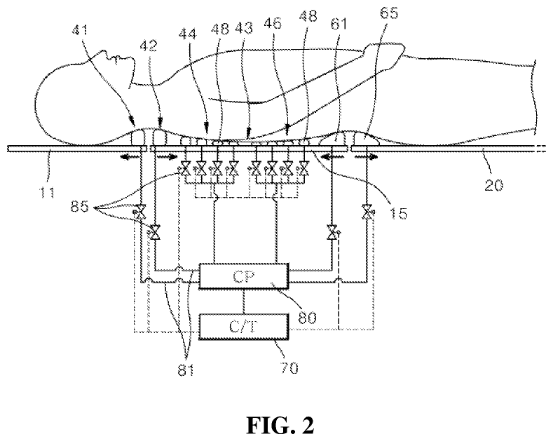

Further, referring to FIGS. 1 and 2, since the respective air bags 48 of the cervical/thoracic relief unit 40, the first and second upper cervical traction tubes 41a and 41b, and the first and second lower cervical traction tubes 42a and 42b are individually connected to the controller, it is possible to support the spine or twist and support the spine by supplying different amounts of air into the respective air bags of the first to fourth thoracic relief units in the extension direction of the spine.

In addition, the cervical/thoracic relief unit may twist and support the cervical vertebrae by supplying the air into the first upper cervical relief tube 41a and the second lower cervical relief tube 42b and by restricting the supply of air into the second upper cervical relief tube 41b and the first lower cervical relief tube 42a. On the contrary, the cervical/thoracic relief unit may twist and support the cervical vertebrae by restricting the supply of air into the first upper cervical relief tube 41a and the second lower cervical relief tube 42b and by supplying the air into the second upper cervical relief tube 41b and the first lower cervical relief tube 42a.

The lateral upper body pressing unit 50 is installed to be movable in the direction of the spine of the upper body in order to press the lateral portion of the upper body supported on the upper body support unit 10 and adjust a curvature of the spine with scoliosis. The lateral upper body pressing unit 50 may include a left upper body pressing unit 51 configured to press a left portion of the upper body, and a right upper body pressing unit 55 configured to press a right portion of the upper body in a direction opposite to a pressing direction of the left pressing unit 51.

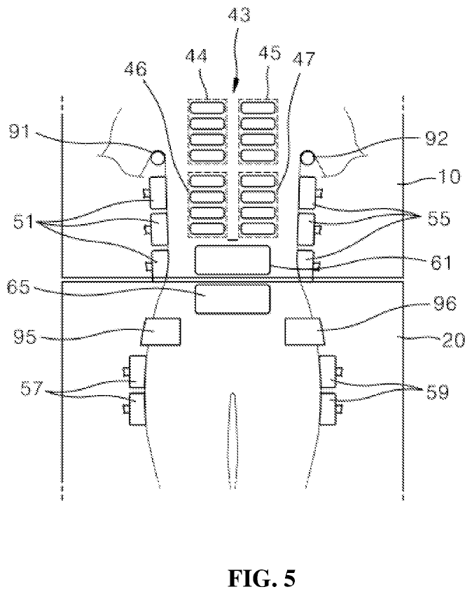

As illustrated in FIG. 5, the plurality of left upper body pressing units 51 and the plurality of right upper body pressing units 55 may be installed in the extension direction of the spine.

The lateral lower body pressing unit 56 includes a left lower body pressing unit 57 and a right lower body pressing unit 59 connected to the controller and configured to press both sides of the lower body under control of the controller in order to correct the pelvis and fix the lower body. As illustrated in FIG. 5, the plurality of lateral lower body pressing units 56 may also be installed in the extension direction of the spine.

The lateral upper body pressing unit 50 and the lateral lower body pressing unit 56 may have a plurality of pneumatic cylinders connected to the compressor 80 and configured to move the left upper body pressing unit 51, the right upper body pressing unit 55, the left lower body pressing unit 57, and the right lower body pressing unit 59 in opposite directions. The present invention is not limited to the configuration in which the lateral upper body pressing unit 50 and the lateral lower body pressing unit 56 are moved by the pneumatic cylinders. Although not illustrated, the lateral upper body pressing unit 50 and the lateral lower body pressing unit 56 may, of course, be installed to be movable by a hydraulic cylinder into which a fluid is introduced by a pump that stores the fluid or a rack gear and a pinion gear that engage with each other.

Referring to FIG. 2, the first and second lumbar traction tubes 61 and 65 are installed at the lower side of the upper body support unit 10 and the upper side of the lower body support unit 20, respectively, which are adjacent to each other in order to press the lumbar vertebrae. The first and second lumbar traction tubes 61 and 65 are connected to the compressor 80 and the air is injected into the first and second lumbar traction tubes 61 and 65 under control of the controller 70 in order to maintain the curvature of the spine when the upper body support unit 10 and the lower body support unit 20 move away from each other in order to stretch the lumbar vertebrae.

Based on the state of the curvature of the spine detected by the spine scanning unit 30, the controller 70 adjusts the amount of air to be supplied into the plurality of air bags 48 and the first and second lumbar traction tubes 61 and 65, the movement distances of the lateral upper body pressing unit 50 and the lateral lower body pressing unit 56, and the spacing distances between the upper body support unit 10 and the lower body support unit 20.

The controller 70 may have a computerized processing unit (not illustrated) that collects signal values detected by the displacement sensor 37 and calculates information that allows the state of the curvature of the spine to be recognized.

The controller 70 may restrict the movement distances of the lateral upper body pressing unit 50 and the lateral lower body pressing unit 56 by controlling the amount of air to be supplied into the plurality of air bags 48 and the first and second lumbar traction tubes 61 and 65 by controlling the plurality of on-off valves 85 that opens or closes the plurality of air supply pipes 81, respectively, which connects the compressor 80, the plurality of air bags 48, the lateral upper body pressing unit 50, the lateral lower body pressing unit 56, and the first and second lumbar traction tubes 61 and 65.

In addition, based on information about the curvature of the spine detected by the spine scanning unit, the controller 70 controls a movement interval between the upper body support unit 10 and the lower body support unit 20 and a movement interval between the head support unit 11 and the thoracic support unit 15. Although not illustrated, there may be provided a first operating unit configured to adjust the movement interval between the upper body support unit 10 and the lower body support unit 20, and a second operating unit configured to manually adjust the movement interval between the head support unit 11 and the thoracic support unit 15. The first and second operating units may be connected to moving means such as a rack gear and a pinion gear by the controller.

The compressor 80 is connected to the plurality of air supply pipes 81 and the controller 70 and supplies the air into the plurality of air bags 48, the first and second lumbar traction tubes 61 and 65, the pneumatic cylinder of the lateral upper body pressing unit 50, and the pneumatic cylinder of the lateral lower body pressing unit 56. Although not illustrated, the compressor 80 may be provided with a storage tank for storing air.

The power supply unit 90 serves to supply power required for the scanning operation of the spine scanning unit 30, the control operation of the controller 70, the operation of the compressor 80, and the opening/closing operation of the on-off valve 85, and has a power switch (not illustrated) connected to the controller 70 and configured to control a supply of commercially available power.

In addition, the spinal traction device according to the present invention may include: the upper body support unit 10 configured to support an upper body of a person; the lower body support unit 20 configured to support a lower body of the person and having the moving means so as to move away from or close to the upper body support unit 10; and the controller 70 configured to adjust the movement of the lower body support unit 20, in which at least one of the upper body support unit 10 and the lower body support unit 20 is installed to be movable upward and downward, such that when the lower body support unit 20 moves, at least one of the upper body support unit 10 and the lower body support unit 20 may move upward or downward in order to press the spine of the upper body.

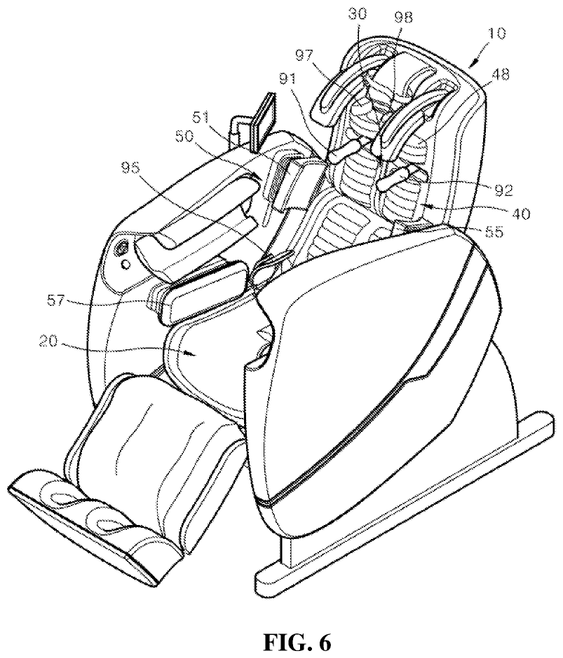

The spinal traction device according to the present invention may be in the form of a bed or in the form of a chair in which the upper body support unit 10 is rotatable to be spaced apart from the lower body support unit 20, as illustrated in FIG. 6.

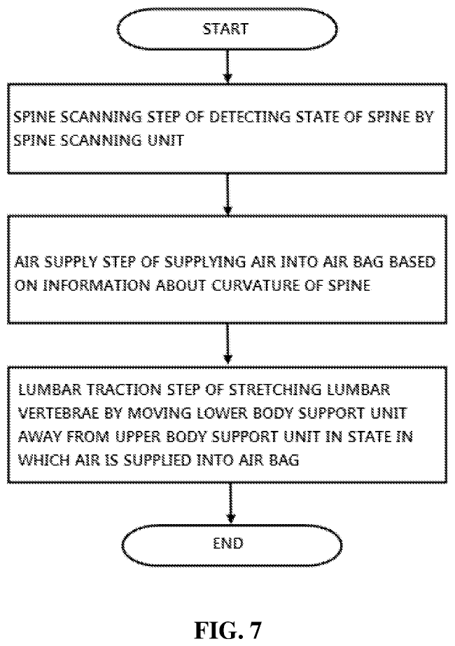

FIG. 7 is a flowchart of a spinal traction method according to the exemplary embodiment of the present invention. Referring to FIG. 7, the spinal traction method according to the present invention may include: a spine scanning step of detecting a state of a spine by the spine scanning unit installed on the upper body support unit for supporting the upper body and configured to be movable in accordance with a curvature of the spine in the direction from the cervical vertebrae to the lumbar vertebrae; an air supply step of supplying air into at least one air bag installed on the upper body support unit by receiving information about the curvature of the spine transmitted from the spine scanning unit; and a lumbar traction step of stretching the lumbar vertebrae by moving the lower body support unit away from the upper body support unit in a state in which the air is supplied into the air bag, and the spinal traction method may include a lateral upper body pressing step between the air supply step and the lumbar traction step, as necessary.

The spine scanning step is a step of detecting the state of the spine by the spine scanning unit 30 installed on the upper body support unit 10 for supporting the upper body and configured to be movable in accordance with the curvature of the spine in the direction from the cervical vertebrae to the lumbar vertebrae. When the power switch of the power supply unit 90 is turned on, the detection roller 31 of the spine scanning unit 30 comes into contact with the portion corresponding to the spine of the upper body and moves in accordance with the curvature of the spine in the direction from the cervical vertebrae to the lumbar vertebrae.

The air supply step is performed after the spine scanning step. The air supply step is a step of receiving the information about the curvature of the spine transmitted from the spine scanning unit 30, supplying the air into the plurality of air bags 48 arranged on the upper body support unit 10 in the extension direction of the spine and installed symmetrically based on the spine scanning unit 30, and supplying the air into the first and second lumbar traction tubes 61 and 65 installed at the lower side of the upper body support unit 10 and the upper side of the lower body support unit 20 for supporting the lower body, respectively, in order to support the lumbar vertebrae.

When the information about the curvature of the spine is transmitted to the controller 70 from the spine scanning unit 30, the controller 70 compares the information with information about a normal curvature of the spine and controls the amount of air to be supplied into the plurality of air bags 48 and the first and second lumbar traction tubes 61 and 65. In this case, the information about the normal curvature of the spine may be a predetermined value set in the controller.

The lateral upper body pressing step is performed after the air supply step. The lateral upper body pressing step is a step of pressing the lateral portion of the upper body by moving, in the direction of the spine of the upper body, the lateral upper body pressing unit 50 installed to press the lateral portion of the upper body supported on the upper body support unit 10 in a direction intersecting a longitudinal direction of the spine based on the information about the curvature of the spine transmitted from the spine scanning unit 30.

The lumbar traction step is a step of stretching the lumbar vertebrae by allowing the upper body support unit 10 and the lower body support unit 20 to move away from each other in the state in which the air is supplied into the first and second lumbar traction tubes 61 and 65 and the lateral portion of the upper body is pressed by the lateral upper body pressing unit 50.

The air supply step is characterized by stretching and correcting the spine by symmetrically, semi-symmetrically, or asymmetrically supporting the spine based on the spine scanning unit by supplying different amounts of air into the respective air bags 48 of the cervical/thoracic relief unit 40 and the first and second lumbar traction tubes 61 and 65 by the controller connected to the cervical/thoracic relief unit 40 and the first and second lumbar traction tubes 61 and 65.

The air supply step includes a cervical pressing step of pressing the cervical vertebrae by supplying the air into the upper and lower cervical relief units 41 and 42, a lumbar pressing step of pressing the lumbar vertebrae by supplying the air into the first and second lumbar traction tubes 61 and 65, and a thoracic support step of supporting the thoracic vertebrae by supplying the air into the first to fourth thoracic relief units 44, 45, 46, and 47.

The thoracic support step performs any one of an operation of supporting the thoracic vertebrae by symmetrically supplying the air, based on the spine scanning unit 30, into the plurality of air bags 48 of the first to fourth thoracic relief units, an operation of semi-symmetrically supporting the thoracic vertebrae by supplying the air to the first thoracic relief unit 44 and the fourth thoracic relief unit 47 and by restricting the supply of air to the second thoracic relief unit 45 and the third thoracic relief unit 46, an operation of semi-symmetrically supporting the thoracic vertebrae by supplying the air to the second thoracic relief unit 45 and the third thoracic relief unit 46 and by restricting the supply of air to the first thoracic relief unit 44 and the fourth thoracic relief unit 47, an operation of asymmetrically supporting the thoracic vertebrae by supplying the same amount of air to the first thoracic relief unit 44 and the third thoracic relief unit 46 and by restricting the supply of air to the second thoracic relief unit 45 and the fourth thoracic relief unit 47, and an operation of asymmetrically supporting the thoracic vertebrae by supplying the air to the second thoracic relief unit 45 and the fourth thoracic relief unit 47 and by restricting the supply of air to the first thoracic relief unit 44 and the third thoracic relief unit 46.

The cervical pressing step, the thoracic support step, and the lumbar pressing step of the air supply step may be performed sequentially or in reverse order. The thoracic support step of the air supply step may be performed in the state in which the lumbar vertebrae and the cervical vertebrae are pressed, or only the thoracic support step may be individually performed. In addition, the cervical pressing step or the lumbar pressing step of the air supply step may be performed after the thoracic support step. In addition, the air supply step may further include a lumbar/cervical pressing step of simultaneously pressing the lumbar vertebrae and the cervical vertebrae. Further, the cervical pressing step and the lumbar pressing step may also press the cervical vertebrae and the lumbar vertebrae, respectively, by supplying different amounts of air into the plurality of air bags.

A cervical traction step is a step of stretching the cervical vertebrae by allowing the head support unit 11 and the thoracic support unit 15 to move away from each other after the air supply step.

The cervical traction step may stretch the cervical vertebrae after the cervical pressing step, the thoracic support step, and the lumbar pressing step in which the air is supplied into the plurality of air bags of the cervical/thoracic relief unit. However, the cervical traction step may also stretch the cervical vertebrae after the lumbar pressing step in which the air is injected only into the upper cervical pressing unit and lower cervical pressing unit.

That is, according to the spinal traction/correction method using the spinal traction/correction device, the lumbar traction step and the cervical traction step may be performed sequentially or in reverse order after the air supply step. Meanwhile, the spinal traction/correction method using the spinal traction/correction device may further include a lumbar/cervical traction step of simultaneously performing the lumbar traction and the cervical traction after the air supply step.

In addition, the spinal traction/correction method according to the present invention may further include a lateral lower body pressing step after the lateral upper body pressing step. The lateral lower body pressing step is a step of moving, in opposite directions, the left lower body pressing unit 57 and the right lower body pressing unit 59 which are connected to the controller and may press both sides of the lower body under control of the controller in order to correct the pelvis and fix the lower body.

As described above, the spinal traction method according to the present invention may press only the cervical vertebrae and the lumbar vertebrae by supplying different amounts of air into the plurality of air bags and the first and second lumbar traction tubes, and may stretch and correct the thoracic vertebrae by twisting or asymmetrically supporting the thoracic vertebrae in the state in which the cervical vertebrae and the lumbar vertebrae are pressed, and as a result, it is possible to improve efficiency in treating spinal stenosis and scoliosis.

In addition, the spinal traction method according to the present invention may detect the state of the curvature of the spine and stretch and correct the spine by supplying different amounts of air into the plurality of air bags and the first and second lumbar traction tubes based on the state of the curvature of the spine, and as a result, there is an advantage of enabling stepwise therapy. In addition, the device and the method for stretching and correcting the spine in accordance with the curvature of the spine according to the present invention may stretch the lumbar vertebrae in the state in which the air is supplied into the first and second lumbar traction tubes, and as a result, there is an advantage of improving efficiency in treating the spine.

While the specific exemplary embodiments according to the present invention have been described above, various modifications may be made without departing from the scope of the present invention. Therefore, the scope of the present invention should not be limited to the described exemplary embodiments and should be defined by not only the claims to be described below, but also those equivalent to the claims.

### DESCRIPTION OF MAIN REFERENCE NUMERALS OF DRAWINGS

10: Upper body support unit
20: Lower body support unit
30: Spine scanning unit
40: Cervical/thoracic relief unit
50: Lateral upper body pressing unit
61: First lumbar traction tube
65: Second lumbar traction tube
70: Controller
80: Compressor
81: Air supply pipe

1. A spinal traction device comprising:
an upper body support unit configured to support an upper body of a person and having at least one spine pressing unit installed on one surface thereof;
a lower body support unit configured to support a lower body of the person and having a moving means so as to move away from or close to the upper body support unit; and
a controller configured to adjust a movement of the lower body support unit,
wherein the at least one spine pressing unit is in close contact with and supports a spine of the upper body when the lower body support unit moves.

2. The spinal traction device of claim 1, wherein the upper body support unit includes:
a head support unit configured to support a head of the person; and
a thoracic support unit configured to support thoracic vertebrae of the person,
wherein at least one of the head support unit and the thoracic support unit is provided with the moving means so that the head support unit and the thoracic support unit move away from or close to each other, and
wherein at least one of the head support unit and the thoracic support unit is provided with the spine pressing unit, such that the spine pressing unit is in close contact with and supports the spine (cervical vertebrae) of a neck part of the upper body when at least one of the head support unit and the thoracic support unit moves.

3. The spinal traction device of claim 1, wherein the spine pressing unit includes a plurality of air bags, and the plurality of air bags is disposed in a longitudinal direction of the spine.

4. The spinal traction device of claim 1, comprising:
a lateral upper body pressing unit installed to be movable in a direction of the spine of the upper body and configured to adjust a curvature of the spine in order to press a lateral portion of the upper body supported on the upper body support unit.

5. The spinal traction device of claim 1, comprising:
first and second lumbar traction tubes installed at an end of the upper body support unit and an end of the lower body support unit, respectively, and configured to be supplied with air from a compressor in order to press lumbar vertebrae.

6. A spinal traction device comprising:
an upper body support unit configured to support an upper body of a person;
a lower body support unit configured to support a lower body of the person and having a moving means so as to move away from or close to the upper body support unit; and
a controller configured to adjust a movement of the lower body support unit,
wherein at least one of the upper body support unit and the lower body support unit is installed to be movable upward and downward, such that the at least one of the upper body support unit and the lower body support unit moves upward or downward to press a spine of the upper body when the lower body support unit moves.

7. A spinal traction method comprising:
a spine scanning step of detecting a state of a spine by a spine scanning unit installed on an upper body support unit for supporting an upper body and configured to be movable in accordance with a curvature of the spine in a direction from cervical vertebrae to lumbar vertebrae;
an air supply step of supplying air into at least one air bag installed on the upper body support unit by receiving information about the curvature of the spine transmitted from the spine scanning unit; and
a lumbar traction step of stretching the lumbar vertebrae by moving a lower body support unit away from the upper body support unit in the state in which the air is supplied into the air bag.
